(19) United States
(12) Patent Application Publication
(10) Pub. No.: US 2021/0346430 A9
(43) Pub. Date: Nov. 11, 2021

(54) METHOD FOR TREATING GLIOBLASTOMA

(71) Applicant: Oaiscell Biotechnologies, Potomac, MD (US)

(72) Inventors: Zhengping Zhuang, Bethesda, MD (US); Kaiyong Yang, Potomac, MD (US); Jing Cui, Potomac, MD (US); Qi Zhang, Hangzhou, Zhejiang (CN)

(21) Appl. No.: 16/652,230

(22) PCT Filed: Aug. 23, 2019
(86) PCT No.: PCT/US2019/048040

§ 371 Date: Mar. 30, 2020

Related U.S. Application Data
(60) Provisional application No. 62/722,959, filed on Aug. 26, 2018.

(51) Int. Cl. A61K 35/17; C07K 14/705; C12N 9/88; A61P 35/00

(52) U.S. Cl. CPC A61K 35/17; A61P 35/00; C12N 9/88; C07K 14/705

(57) ABSTRACT

Disclosed is a method for treating glioblastoma or other brain tumors. The method includes steps of preparing cells comprising a chimeric antigen receptor (CAR) molecule and administering to a mammal in need thereof an effective amount of the prepared cells. Also disclosed is that the CAR molecule contains an antigen binding domain that binds to the tumor antigen associated with the glioblastoma or other brain tumors, and the tumor antigen is carbonic anhydrase IX.

### CROSS-REFERENCE TO RELATED APPLICATIONS

This application is a U.S. National Stage Entry of International Application No. PCT/US2019/048040, filed on Aug. 23, 2019, which claims the benefit of U.S. Provisional Application Ser. No. 62/722,959, filed Aug. 26, 2018. All of the foregoing applications are incorporated by reference herein in their entireties.

### STATEMENT AS TO RIGHTS TO INVENTIONS MADE UNDER FEDERALLY SPONSORED RESEARCH AND DEVELOPMENT

This invention was made with Government support under project number Z01BC011773-01 by the National Institutes of Health, National Cancer Institute. The Government has certain rights in the invention.

### FIELD OF THE INVENTION

The present invention relates generally to the use of immune effector cells (e.g., T cells, NK cells) engineered to express a chimeric antigen receptor to treat a disease associated with expression of a tumor antigen.

### BACKGROUND OF THE INVENTION

Glioblastoma (GBM) is the most common malignant brain tumor in humans. Also known as glioblastoma multiforme, glioblastoma is one of a group of tumors called astrocytomas. It typically starts in astrocytes, star-shaped cells that nourish and support nerve cells in the brain. Surrounded by a lot of blood vessels that feed it, glioblastoma grows very fast inside the brain. Glioblastoma is the most common malignant primary brain tumor diagnosed in adults, with an estimated 12,000-13,000 new cases occurring each year in the United States.

Glioblastoma has a poor prognosis. Currently only tumor resection, radiotherapy, and temozolomide chemotherapy might show clinical benefits to some degree for patients with glioblastoma. Yet, survival generally ranges only about 14-18 months, and the 5-year survival rate is less than 10%.

Thus, there is an urgent need to develop novel treatments that promote survival.

Chimeric antigen receptor (CAR) T therapy emerged recently as the most important advance in the cancer field as nominated by the American Society of Clinical Oncology (see Clinical Cancer Advances 2018: Annual Report on Progress Against Cancer from the American Society of Clinical Oncology. J Clin Oncol 2018:JCO2017770446). To date, two commercial CAR T products, including Tisagenlecleucel (also known Kymariah) from Novartis and Axicabtageneciloleucel (also known Yescarta) from Kite Pharma, have been approved by the US Food and Drug Administration for the treatment of acute lymphoid leukemia. Given their extraordinary efficacy in hematological malignancies, efforts have been made to apply CAR T therapies to solid tumors. However, the field is still in its infancy and more positive clinic outcomes are needed to validate the approach. On the other hand, different from other solid tumors, glioblastoma often presents a unique set of challenges for developing an effective therapy.

### SUMMARY OF THE INVENTION

This invention provides a CAR T therapy-based method for treating glioblastoma. During the study, the method, unexpectedly, exhibited significant benefits to clinic subjects with glioblastoma.

One aspect of this invention relates to a method for treating glioblastoma or other brain tumors, the method includes steps of (i) preparing cells comprising a chimeric antigen receptor (CAR) molecule, and (ii) administering to a mammal in need thereof an effective amount of the prepared cells. The CAR molecule contains an antigen binding domain that binds to the tumor antigen associated with the glioblastoma or other brain tumors and the tumor antigen can be carbonic anhydrase IX.

Particularly, the administering of the prepared cells is through intracranial injection and the intracranial injection is directly targeting within a boundary of the glioblastoma or the other brain tumors.

The intracranial injection is, preferably, conducted stereotactically.

In the above-described method, the prepared cells can further include an agent for use in combination with the CAR molecule to increase the efficacy of the treatment.

In one embodiment, the agent can be a molecule stimulating lymphocyte proliferation. Examples of such molecule include interleukin 7.

In another embodiment, the agent can be a molecule recruiting through chemotaxis endogenous immune cells to eliminate glioblastoma and other brain tumors. Examples of the molecule include chemokine (C-C motif) ligand 19.

Further, the method of this invention can include additional steps. One example of the steps is, before the administering of the prepared cells, treating the glioblastoma or the other brain tumors with a therapy that inhibits vascular endothelial growth factor. Specifically, a therapy that inhibits vascular endothelial growth factor can be administering an effective amount of Bevacizumab (Avastin).

The method of this invention can be applied to a mammal, e.g., a human or a mouse.

The details of the invention are set forth in the drawing and the description below. Other features, objects, and advantages of the invention will be apparent to those persons skilled in the art upon reading the drawing and the description, as well as from the appended claims.

### BRIEF DESCRIPTION OF THE DRAWING

FIGS. 1A-1D illustrate that CAIX is highly expressed in patients with glioblastoma and human glioblastoma cell lines under hypoxic conditions. FIG. 1A: Representative images of CAIX immunohistochemical staining exhibit CAIX expression in tumoral areas of human glioblastoma samples. FIG. 1B: Western blot showed high expression of CAIX in the glioblastoma cell lines (A172, LN229, T98G and U251) and glioma stem cell line GSC 923 after hypoxia treatment (1% O₂) for 48-hours, but not in glioma stem cell line GSC827. ACTIN was used as a loading control (N, normoxia; H, hypoxia). FIG. 1C: Flow cytometry analysis showed CAIX cell surface expression in hypoxia (yellow) treated glioblastoma cell lines, compared to normoxia treated ones (blue). FIG. 1D: Representative images of immunohistochemistry staining pattern of CAIX in xenograft U251 tumors at d7, d14, and d21. Enhanced expression of CAIX was observed during tumor growth. Scale bar, 60 μm.

FIGS. 2A and 2B show that CAIX is highly expressed in tumor cells in patients with glioblastoma. FIG. 2A: Representative western blots show CAIX expression in human glioblastomas. ACTIN was used as a loading control. FIG. 2B: Immunostaining detected CAIX expression in tumor cells, but not in T cells (CD3), endothelial cells (CD31), or macrophages (IBA1). White box in the upper right corner showed the magnified area. Scale bar, 20 μm.

FIGS. 3A and 3B illustrate that RNA expression of CAIX with relation to survival data in TCGA and GTEx databases. FIG. 3A: The RNA-seq expression level of CAIX in glioblastomas is significantly higher than normal brain tissue (*p<0.05). TPM, Transcriptional per million. FIG. 3B: Kaplan-Meier survival curves of patients with glioblastoma stratified by high and low CAIX expression. The low CAIX expression group (blue line) has a significantly better overall survival compared with the high CAIX expression group (red line, p<0.05).

FIGS. 4A and 4B illustrate the generation of a CAIX-overexpressed glioblastoma cell line. FIG. 4A: Western blots showed high expression of CAIX in U251 cells transfected with CAIX-HA (CAIX+U251). Anti-HA tag antibody was used to confirm CAIX expression in CAIX transfected cell lines. #4, 5, 6 clones are CAIX+ clones. FIG. 4B: Flow cytometry analysis showed CAIX cell surface expression CAIX in CAIX+U251 cells. Expression level of CAIX in CAIX+ cells (green) is comparable to that in hypoxia-treated U251 naïve cells (yellow). Unstained U251 cells (red) and U251 cells (blue) cultured in normoxia served as negative control.

FIGS. 5A-5E show that generation and in vitro cytotoxic activity of anti-CAIX CAR-T cells. FIG. 5A: Scheme of CAIX-specific chimeric antigen receptors (CAR) design. Anti-CAIX CAR was generated by cloning a single chain variable fragment (scFv) of CAIX antibody into a lentiviral vector containing CD8 hinge, a CD28 transmembrane domain, and CD28, 4-1BB, and CD3ζ intracellular signaling domains. FIG. 5B: Transduction efficiency was detected by GFP expression in mock T cells on day 6 post-transduction using flow cytometry. The transduction efficiency was around 30%. FIG. 5C: T cells (effector) were co-incubated with tumor cells (target) for 48 hours at different effector (E):target (T) ratios. Cytotoxicity was measured by LDH release assay (N=4). CAIX-transfected U251 (CAIX+) cells had more significant response to anti-CAIX CAR-T cells. While higher E/T ratio (5/1) showed more significant cytotoxicity. Each data point is the mean±SEM of 4 replicates. FIG. 5D: Naïve U251 or CAIX+U251 cells were pretreated in normoxia or hypoxia (1% 02) for 24-hours. Control T or anti-CAIX CAR-T cells were co-cultured with tumor cells at an E/T ratio of 4 for 48 hours. Cytotoxicity was measured using naïve U251 or CAIX+U251 cells in normoxia and hypoxia by LDH release assay (N=4). A higher number of tumor cells showed significantly increased cytotoxicity of CAR-T cells. FIG. 5E: Secreted cytokine (IFN-γ, IL-2, TNF-α) levels in supernatant were measured by ELISA. All data are shown as the mean±SEM. *p<0.05, **p<0.01, and***p<0.001 by Student's t test.

FIGS. 6A-6B illustrate that generation and in vitro cytotoxic activity of anti-CAIX CAR-T cells. FIG. 6A: T98G or LN229 cells were pretreated in normoxia or hypoxia (1% 02) for 24-hours. Control T or anti-CAIX CAR-T cells were co-cultured with tumor cells at an E/T ratio of 4 for 48 hours. Cytotoxicity was measured by LDH release assay (N=4). The bar graphs showed that hypoxia increased cytotoxicity of anti-CAIX CAR-T cells. FIG. 6B: Secreted cytokine (IFN-γ, IL-2, TNF-α) levels in supernatant were measured by ELISA. All data are shown as the mean±SEM. *p<0.05, **p<0.01, and ***p<0.001 by Student's t test.

FIGS. 7A-7C show that cytotoxicity of anti-CAIX CAR-T is antigen dependent. FIG. 7A: Western blots showed CAIX expression was undetectable in CAIX knockout U251 cells using CRISPR/Cas9 after hypoxia treatment (1% 02) for 48-hours. KO #1 and #2 were two independent CAIX knockout clones. FIGS. 7B and 7C: Cells expressing CAIX (U251 naïve cells and GSC923) and cells lacking CAIX expression (CAIX knockout cells and GSC827) were pretreated in hypoxia (1% 02) for 24-hours. Control T or anti-CAIX CAR-T cells were co-cultured with tumor cells at an E/T ratio of 4 for 48 hours. Cytotoxicity was measured by LDH release assay (N=4). The bar graphs showed that hypoxia increased cytotoxicity of anti-CAIX CAR-T cells in U251 naïve cells (B) and GSC923 cells (C) but not in CAIX knockout cells (B) and GSC827 cells (C). All data are shown as the mean±SEM. ***p<0.001 by Student's t test.

FIGS. 8A-8D illustrate that anti-CAIX CAR-T cells significantly suppress tumor growth in glioblastoma. FIG. 8A: The schematic diagram of the progression of experiment in vivo. NSG mice received intracranial injection of 1×10⁵ U251-luc cells on day 0. On day 7, the tumors were imaged and mice were randomized into 3 groups: un-treated (N=8), control T (N=9) and anti-CAIX CAR-T cell treated group (N=10). Tumors were treated by intra-tumoral administration of 3 doses (every week) of 2×10⁶ control or anti-CAIX CAR-T cells. FIG. 8B: Bioluminescence imaging was used to follow tumor progression. The luminescence signal showed reduced U251-luc tumor burden compared with the untreated group and control T group. p value was calculated by two-way ANOVA. ***p<0.001. FIG. 8C: Survival curve showed mice treated with anti-CAIX CAR-T cells had a significantly prolonged survival compared with the untreated group and control T group. p value was calculated by long-rank test analysis. *** p<0.001. The median survival of anti-CAIX CAR-T treated group was 66.5 days, while that was 39.5 days and 41 days in un-treated group and control T treated group respectively. Two out of ten (20%) anti-CAIX CAR-T treated mice were cured. FIG. 8D: The tumor-derived bioluminescence images of two cured mice showed a complete response induced by anti-CAIX CAR-T cells on day 9.

FIG. 9 shows gating strategy for flow cytometric analysis of tumor infiltrating lymphocytes. We first used SSC-FSC gate to exclude no cellular debris, followed by exclusion of duplets by FSC-H-FSA-A gated. Live-dead stain was used to exclude dead cells. Live cells were then gated based on expression of GFP+ tumor cell marker. GFP− cells were considered as non-tumor cells including leukocytes. GFP− cells were then phenotyped further based on CD3, CD4 and CD8 expression. CD3+CD4+ cells were gated as CD4+ lymphocytes, while CD3+CD8+ cells were gated as CD8+ lymphocytes.

FIGS. 10A-10F illustrate that targeting CAIX produces a robust CAR-T cell response. FIGS. 10A-10C: TILs analysis for glioblastoma xenograft mouse model established as described above. Mice were randomized into three groups: un-treated (N=6), control T (N=5), and anti-CAIX CAR-T (N=4). U251-luc tumors in the respective groups were harvested two weeks after initiation of treatment and analyzed by flow cytometry. Representative FACS plots of CD4+ and CD8+ cells in tumors (A). Percentage of CD3+ T cells in tumors (B). Percentage of CD4+ and CD8+ cells in tumors (C). FIG. 10D: Flow cytometry analysis showed percentage of CD4+ and CD8+ cells in control T cells and CAR-T cells before injection. The bar graphs represent a high amount of cytotoxic CD8+ T cells in both groups, while the ratio of CD4+ and CD8+ T cells are comparable between two groups before injection. FIGS. 10 E-10F: Cytokine (IFN-γ, TNF-α and IL-2) secretion in the supernatant of tumor (E) and blood (F) was analyzed by ELISA. The bar graphs represent a significant increase of cytokine release in anti-CIX CAR-T treated groups. All data are shown as the mean±SEM. *p<0.05, **p<0.01, and ***p<0.001 by Student's t test, anti-CAIX CAR-T group vs. un-treated group or control T group.

FIGS. 11A-11B show that combination of Avastin and anti-CAIX CAR-T cells synergistically suppress tumor growth in glioblastoma xenograft mouse model. FIG. 11A: The schematic diagram of the progression of experiment in vivo. One week after 1×10⁵ U251-luc cells were inoculated into the brain of NSG mice, mice were randomized into four groups (N=6 for each group): un-treated, Avastin, anti-CAIX CAR-T, and Combo (Avastin plus anti-CAIX CAR-T). Mice in anti-CAIX CAR-T and Combo treated groups were injected in situ with 2×10⁶ anti-CAIX CAR-T cells. Avastin was administrated into mice in Avastin and Combo groups twice every week at a dose of 10 mg/kg until survival endpoint. Mice were monitored every four days for 16 days via luminescence imaging to follow tumor progression. FIG. 11B: Bioluminescence imaging results showed that the combination of Avastin resulted in striking regression of tumors compared to Avastin or anti-CAIX CAR-T alone group. p value was calculated by two-way ANOVA. **p<0.01.

### DETAILED DESCRIPTION

Methods are provided for treating a subject having glioblastoma or other types of brain tumors. Aspects of the methods include administering to the individual CAR-T cells specific for carbonic anhydrase IX (CAIX) in an amount effective to destroy the tumors. Also provided are reagents including bio-engineered products that find use in practicing the subject methods.

Before the present methods are described, it is to be understood that this invention is not limited to a particular method described, as such may, of course, vary. It is also to be understood that the terminology used herein is for the purpose of describing particular embodiments only, and is not intended to be limiting, since the scope of the present invention will be limited only by the appended claims.

The subject methods are useful primarily for therapeutic purposes. Thus, as used herein, the term “treating” is used to refer to both prevention of disease, and treatment of a pre-existing condition. The treatment of ongoing disease, to stabilize or improve the clinical symptoms of the patient, is a particularly important benefit provided by the present invention. Such treatment is desirably performed prior to loss of function in the affected tissues including the central nervous system and its surrounding tissues. For example, treatment of a cancer patient may be reduction of tumor size, elimination of malignant cells, or the prevention of relapse in a patient who has been put into remission.

The terms “inhibiting,” “reducing,” or “prevention,” or any variation of these terms, when used in the claims and/or the specification includes any measurable decrease or complete inhibition to achieve a desired result.

On the other hand, the terms “determining,” “measuring,” and “assessing,” and “assaying” are used interchangeably and include both quantitative and qualitative determinations.

The terms “subject,” “host,” “patient,” and “individual” are used interchangeably herein to refer to any mammalian subject for whom diagnosis or therapy is desired, particularly humans. Other subjects may include cattle, dogs, cats, guinea pigs, rabbits, rats, mice, horses, and so on.

The terms “cell,” and “cells,” and “cell population,” used interchangeably, intend one or more mammalian cells. The term includes progeny of a cell or cell population. Those skilled in the art will recognize that “cells” include progeny of a single cell, and there are variations between the progeny and its original parent cell due to natural, accidental, or deliberate mutation and/or change.

The terms “cell proliferation” and “to proliferate” as used herein refer to the amplification of the cell by cell division.

A “cancer cell” as used herein refers to a cell exhibiting a neoplastic cellular phenotype, which may be characterized by one or more of, for example, abnormal cell growth, abnormal cellular proliferation, loss of density dependent growth inhibition, anchorage-independent growth potential, ability to promote tumor growth and/or development in an immunocompromised non-human animal model, and/or any appropriate indicator of cellular transformation. “Cancer cell” may be used interchangeably herein with “tumor cell” or “cancerous cell”, and encompasses cancer cells of a solid tumor, a semi-solid tumor, a primary tumor, a metastatic tumor, and the like.

Immune effector cells are the transiently activated cells that defend the body in an immune response. Once the triggering antigen/pathogen has been cleared, immune effector cells eventually stop proliferating and die. Effector B cells are called plasma cells and secrete antibodies, and activated T cells include cytotoxic T cells and helper T cells.

“Immunotherapy” refers to treatment of disease (e.g., cancer) by modulating an immune response to a disease antigen. In the context of the present application, immunotherapy refers to providing an anti-cancer immune response in a subject by administration of an antibody (e.g., a monoclonal antibody) and/or by administration of an antigen that elicits an anti-tumor antigen immune response in the subject.

The CAR-T cells prepared in the above-described method are substantially enriched or substantially isolated before applying to a subject.

As used herein, the term “substantially enriched” or “substantially isolated” indicates that a cell population is at least about 20-fold, more preferably at least about 500-fold, and even more preferably at least about 5000-fold or more enriched from an original mixed cell population comprising the desired cell population.

The term “antibody” is used interchangeably with “immunoglobulin.” It encompasses polyclonal and monoclonal antibody preparations where the antibody may be of any class of interest (e.g., IgG, IgM, and subclasses thereof), as well as preparations including hybrid antibodies, altered antibodies, F(ab′).sub.2 fragments, F(ab) molecules, Fv fragments, single chain fragment variable displayed on phage (scFv), single chain antibodies, single domain antibodies, diabodies, chimeric antibodies, humanized antibodies, and functional fragments thereof which exhibit immunological binding properties of the parent antibody molecule.

The term “monoclonal antibody” refers to an antibody composition having a homogeneous antibody population. The term is not limited by the manner in which it is made. The term encompasses whole immunoglobulin molecules, as well as Fab molecules, F(ab′)2 fragments, Fv fragments, single chain fragment variable displayed on phage (scFv), fusion proteins comprising an antigen-binding portion of an antibody and a non-antibody protein, and other molecules that exhibit immunological binding properties of the parent monoclonal antibody molecule.

Those skilled in the art understand how to make and screen polyclonal and monoclonal antibodies.

The terms “antigen” and “epitope” are well understood in the art and refer to the portion of a macromolecule (e.g., a polypeptide) which is specifically recognized by a component of the immune system, e.g., an antibody or a T-cell antigen receptor. As used herein, the term “antigen” encompasses antigenic epitopes, e.g., fragments of an antigen which are antigenic epitopes. Epitopes can be recognized by antibodies in solution, e.g. free from other molecules. Epitopes can be recognized by T-cell antigen receptor when the epitope is associated with a class I or class II major histocompatibility complex molecule.

The term “specific binding of an antibody” or “antigen-specific antibody” in the context of a characteristic of an antibody refers to the ability of an antibody to preferentially bind to a particular antigen that is present in a homogeneous mixture of different antigens. In certain embodiments, a specific binding interaction will discriminate between desirable and undesirable antigens (or “target” and “non-target” antigens) in a sample, in some embodiments more than about 10 to 100-fold or more (e.g., more than about 1000- or 10,000-fold). In certain embodiments, the affinity between an antibody and antigen when they are specifically bound in an antibody-antigen complex is characterized by a K.sub.D (dissociation constant) of less than 10.sup.-6M, less than 10.sup.-7 M, less than 10.sup.-8 M, less than 10.sup.-9 M, less than 10.sup.-9 M, less than 10.sup.-11M, or less than about 10.sup.-12M or less.

An “effective amount” is an amount sufficient to effect beneficial or desired clinical results. An effective amount can be administered in one or more administrations. For purposes of this invention, an effective amount of reagent antibodies is an amount that is sufficient to diagnose, palliate, ameliorate, stabilize, reverse, slow or delay the progression of the disease state.

Normally, the blood brain barrier (BBB) encompasses blood vessels that deliver nutrients and oxygen to the brain tissue. Brain tumors cannot metastasize to the organs out of central nervous system because of the BBB. This phenomenon facilitates the intracranial application of CAR-T therapy by limiting the adverse events of CAR-T cell within the whole body. Although some investigators have proven that CAR-T cells injected via peripheral vein could be found in GBM and showed cytotoxicity, the dose of systemic use of CAR-T cells can be several orders higher than that of intracranial application. The required number of CAR-T cells for intracranial injection in a patient is easily to fulfill, even for a multi-injection strategy. The only common way for GBM spread is spinal cord metastasis. Fortunately, intraventricular injection of CAR-T cells was able to control the spinal cord metastases (10.1158/1078-0432.CCR-15-0428). In the study, the complete regression rate of 60%, though the total number was small, was beyond the expectation and proved the CAIX CAR-T itself a promising tool for GBM treatment.

Clinically, surgery is still the first choice for GBM. However, residual tumor cells are always expected because GBM cells can be highly infiltrated, and it is unrealistic to perform an extended resection to keep all malignant cells away. In this case, an intracranial injection of CAR-T cells can be easily conducted during or after surgery to remove residual tumor cells as much as possible. Meanwhile, intracranial injection of CAR-T cells can be performed together to help decrease the risk of tumor cell spread in the central nervous system.

On the other hand, CAIX is a membrane-located protein and functions by maintaining intracellular pH. CAIX is mildly expressed in normal cells and can be induced by hypoxia through hypoxia-inducible factor 1α. Due to the increased glycolytic activity of tumor cells and hypoxia in tumor microenvironment, CAIX is overexpressed and is essential for the survival of tumor cells in various types of cancer. CAIX overexpression is also found to promote tumor progression and is associated with poor prognosis in many cancers.

In renal cell carcinoma, which is characterized by enhanced hypoxia signaling due to frequent loss-of-function of VHL mutations, CAIX-targeted CAR T therapy showed an anti-tumoral effect in a mouse model. A phase I/II trial of CAIX-targeted CAR T for metastatic renal cell carcinoma failed because the patients developed anti-CAR T-cell humoral and cellular immune responses.

Yet a proof-of-concept study was carried out to show the possibility of CAIX as a CAR-T target for GBM. CAIX is an inducible membrane-located protein due to hypoxia or pseudohypoxia. The use CAIX as a target takes advantage of rapid proliferation of GBM. Normal brain tissue and gliomas of grade I to III seem to have a low incidence of CAIX expression. The CAIX detectable GBM in the study was 60-70%, which was similar to a previous study (10.1093/neuonc/nos216). GBM cell lines in normoxia exhibit a low expression of CAIX, but some of them could still be killed by the CAR-T cells. This may be because a relatively local hypoxia induced by oxygen consumption caused by the CAR-T cells, which can also happen in human patients. It was found that the expression of CAIX in naïve U251 cells was up-regulated when co-cultured with T cells (data not shown). Therefore, in case of CAR-T cell injection in human GBM, T cells may further induce CAIX expression in addition to the effect of compromised microvasculature.

Analyses of other CAR-T showed increased infiltration of dendritic cells and T cells into tumor tissues. Depletion of recipient T cells before CAR-T cell administration negatively affects the therapeutic effects of the CAR-T cell treatment, suggesting that CAR-T cells and recipient immune cells collaborated to exert anti-tumor activity.

Further, CAR-T cell therapies can benefit from a combination with other agents, e.g., those that stimulate lymphocyte proliferation or recruit through chemotaxis endogenous immune cells to eliminate tumors. In the method of instant invention, the prepared CAR-T cell can also include interleukin 7 for stimulating lymphocyte proliferation or chemokine (C-C motif) ligand 19 for recruiting through chemotaxis endogenous immune cells comprises.

Combining CAR-T therapy with other treatments has been found to acquire better tumor control. It is believed that combination of CAIX CAR-T with anti-angiogenic agents such as Avastin or sorafenib is supposed to show a better efficacy. On one hand, anti-angiogenesis can lead to hypoxia in tumor microenvironment and induce the expression of CAIX. On the other hand, hypoxia has been reported to enhance the function of cytotoxic T cells.

The term “in combination with” as used herein refers to uses where, for example, a first therapy is administered during the entire course of administration of a second therapy; where the first therapy is administered for a period of time that is overlapping with the administration of the second therapy, e.g. where administration of the first therapy begins before the administration of the second therapy and the administration of the first therapy ends before the administration of the second therapy ends; where the administration of the second therapy begins before the administration of the first therapy and the administration of the second therapy ends before the administration of the first therapy ends; where the administration of the first therapy begins before administration of the second therapy begins and the administration of the second therapy ends before the administration of the first therapy ends; where the administration of the second therapy begins before administration of the first therapy begins and the administration of the first therapy ends before the administration of the second therapy ends. As such, “in combination” can also refer to a regimen involving administration of two or more therapies. “In combination with” as used herein also refers to administration of two or more therapies that may be administered in the same or different formulations, by the same or different routes, and in the same or different dosage form type.

However, the sequence of CAR-T therapy and anti-angiogenic agents may be important and needs further experiments to figure it out. Combinations with other clinical used therapies are also possible if the rationale is clear. For instance, CAR-T therapy is expected to be used together with checkpoint inhibitors since the latter can decrease the exhaustion of CAR-T cells.

### BEST MODE FOR CARRYING OUT INVENTION

The following example explains the present invention more concretely, but do not limit the range of the present invention.

### Example 1

### Cell Culture and Reagents

HEK293T cells and glioblastoma cell lines including U251, LN 229, T98G, and A172 were derived from American Type Culture Collection (ATCC; Manassas, Va.). All cells were cultured in Dulbecco's Modified Eagle Medium (DMEM; Gibco) supplemented with 10% fetal bovine serum (FBS; Gibco) and 1% penicillin and streptomycin (Gibco). U251-luc cells were generated by stable transfection of luciferase-containing lentiviruses (EF1a-ffLuc2-eGFP) into naïve U251 cells.

### Human Sample Acquisition

Frozen glioblastoma tissues were obtained from the tissue bank of Surgical Neurology Branch at National Institute of Neurological Disorders and Stroke (NINDS), National Institutes of Health (NIH; Bethesda, Md.). Formalin-fixed paraffin-embedded glioblastoma tissues were acquired from Huashan Hospital, Fudan University (Shanghai, China).

### Immunoblotting and Immunohistochemistry

Immunoblotting was performed as it follows. In brief, proteins were collected from frozen tissue or cell lines. A total of 40 μg proteins were subjected to electrophoresis and were transferred to a nitrocellulose membrane. After blocking with 5% non-fatty milk, the membrane was incubated with primary antibodies (1:1000 dilution) at 4° C. overnight, followed by incubation of secondary antibodies (1:3000 dilution; from Cell Signal Technology). Anti-CAIX antibody was purchased from Novus Biologicals (Littleton, Colo.).

### Flow Cytometry

Cells were treated as indicated and were harvested. APC-conjugated anti-CAIX antibodies (R&D Systems, Minneapolis, Minn.) were used to stain the cells (1 μg) for 1 hour in the dark according to the manufacture's protocol. 4′,6-diamidino-2-phenylindole (DAPI) was added before cells were subjected to flow cytometry using a BD FACS Canto II Flow Cytometer (BD Biosciences, San Jose, Calif.). Data were analyzed using FlowJo software (FlowJo, Ashland, Oreg.).

### Generation of CAIX CAR-Expressing Vector

The CAIX CAR-expressing vector (Lenti-EF1a-CAIX-3rd-CAR) was generated using the pLenti-EF1a-C-mGFP Tagged Cloning Vector (OriGene Technologies, Rockville, Md.). In brief, the mGFP sequence on the original vector was replaced by the CAR cassette including signal peptide, anti-CAIX single-chain variable fragment (scFv), CD8 hinge, CD28 transmembrane intracellular domain, 4-1BB, and CD3zeta. The final vector was confirmed by restriction digestion and Sanger sequencing.

### Lentivirus Production and Transduction

Lentiviral envelope expressing plasmid pMD2.G and packaging plasmid psPAX2 were Addgene plasmid #12259 and 12260, respectively. pMD2.G, psPAX2, and Lenti-EF1a-CAIX-3rd-CAR plasmids were transfected at a ratio of 2:4:5 into HEK293T cells cultured in DMEM without antibiotics. Medium was changed every day, and the supernatants were collected for the next two days. The lentiviruses were quantified using HIV-1 p24 Antigen ELISA (ZeptoMetrix, Buffalo, N.Y.) and were concentrated using Lenti-X Concentrator (Clontech Laboratories, Mountain View, Calif.).

Peripheral blood mononuclear cells (PBMCs) were derived from healthy donors recruited by the Blood Bank, Clinical Center, NIH and kept in liquid nitrogen until used. PBMCs were thawed in RPMI 1640 overnight and activated with Dynabeads Human T-Activator CD3/CD28 (Thermo Fisher Scientific) at a ratio of 1:1 in AIM V medium (Gibco) supplemented with 5% human serum (Gibco) for 24 hours. Living cells were enriched using lymphocyte separation medium and washed with phosphate buffered saline (PBS; Gibco) twice. T cells were then transduced with lentiviruses containing CAIX CAR vectors or empty vectors at 1200 g for 2 hours at 32° C. in a V-bottom 96-well plate (Corning, Corning, N.Y.). Each well contained 0.25 million cells and viruses at a MOI of 40, with 8 μg/ml polybrene (Sigma-Aldrich) and 300 international units (IU) human interleukin 2 (ML-2; Peprotech, Rocky Hill, N.J.). Transduced cells were resuspended after 3 hours and were transferred to a 6-well plate for expansion in the presence of 100 IU hIL-2 for two to three days.

### Enzyme-Linked Immunoabsorbent Assay (ELISA)

Cells were treated as indicated for 48 hours, and supernatants were collected. Cells and cell debris were removed from samples by centrifugation at 5,000 g for 5 min, and the samples were kept in −80° C. until used. Blood samples from mice were collected into tubes with EDTA from the orbital sinus, and then the blood cells were removed by centrifugation at 10,000 g for 10 min, and the plasma was stored in −80° C. until used.

Concentrations of TNF-α and IFN-γ were determined using Human TNF ELISA Kit II (BD Biosciences, San Jose, Calif.) and Human IFN gamma ELISA Read-SET-Go! (Affymetrix, San Diego, Calif.), respectively, according to the manufacturer's instructions.

### Xenograft Mouse Model

Mice experiments were approved by the NINDS and National Cancer Institute (NCI) Animal Use and Care Committees. NOD-Prkdc^{scid}Il2re^{tmiWjl} (NSG) mice (6-8 weeks old from NCI-Frederick animal facility) were intracranially inoculated with 100,000 U251-luc cells suspended in 2 μL Hank's Balanced Salt Solution (HBSS; Crystalgen, Commack, N.Y.). After one week, luciferin signals were detected to confirm the survival of tumor cells in mice. The mice were assigned to the indicated groups according to the signal intensity to keep the baseline balanced. A total of 2 million anti-CAIX CAR-T cells, or empty vector transduced T cells, or mock (control T cells) in 2-2.5 μL HBSS were injected into the tumors. Untreated mice received injection of the same volume of HBSS. Avastin was intraperitoneally injected twice every week at a dose of 10 mg/kg body weight for 30 days. The viability of tumors was monitored every three days. Survival end point for all animal studies were defined as when any of the following criteria was reached: 1) a loss of more than 15% of body weight, 2) protruded skull, 3) head tile, 4) hunched posture, 5) ataxia, 6) rough hair coat, or 7) impaired mobility.

### Isolation of Tumor-Infiltrating Lymphocytes (TILs)

Mice were intracranially inoculated with 100,000 U251-luc cells suspended in 2 μLHBSS and treated as above after 1 week. Mice were sacrificed, and tumors were excised 3 weeks after treatment. Tumors were subjected to mechanical disruption using a Gentle MACS Dissociator (Miltenyi Biotec, Bergisch Gladbach, Germany) in presence of enzymatic digestion using Tumor Dissociation Kit (Miltenyi Biotec). The supernatant was harvested after a brief spin. Cells and cell debris were further removed from supernatant by centrifugation at 10,000 g for 10 min, and the samples were kept in −80° C. until ELISA analysis.

Suspensions containing T cells were stained with anti-human CD3 (#317332), CD4 (#300514), CD8 (#301032) antibodies (Biolegend, San Diego, Calif.) in FACS buffer and then analyzed by a BD FACS Canto II Flow Cytometer (BD Biosciences, San Jose, Calif.). Data analysis was performed using FlowJo software (FlowJo, Ashland, Oreg.).

### Statistical Analysis

Data were presented as the mean and standard deviation (SD) or standard error of the mean (SEM), as indicated. Survival curves were generated using the Kaplan-Meier estimate. Statistical analysis was performed using Prism 6 (GraphPad Software, San Diego, Calif.). Survival curves were compared using log-rank test. Other variables were analyzed using unpaired Student's t test. A p<0.05 was considered as statistically significant.

### Results

### Overexpression of CAIX in GBM

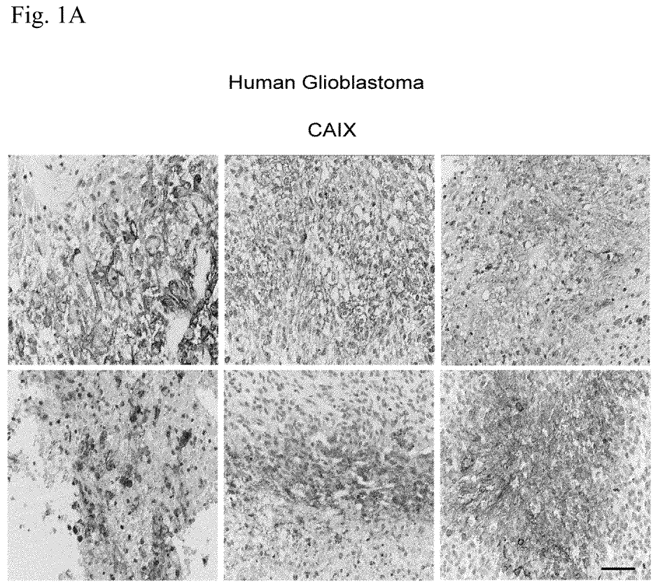

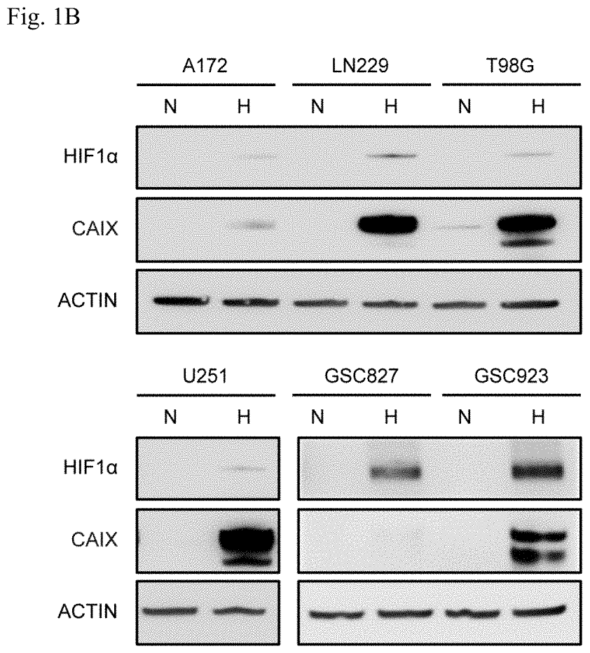

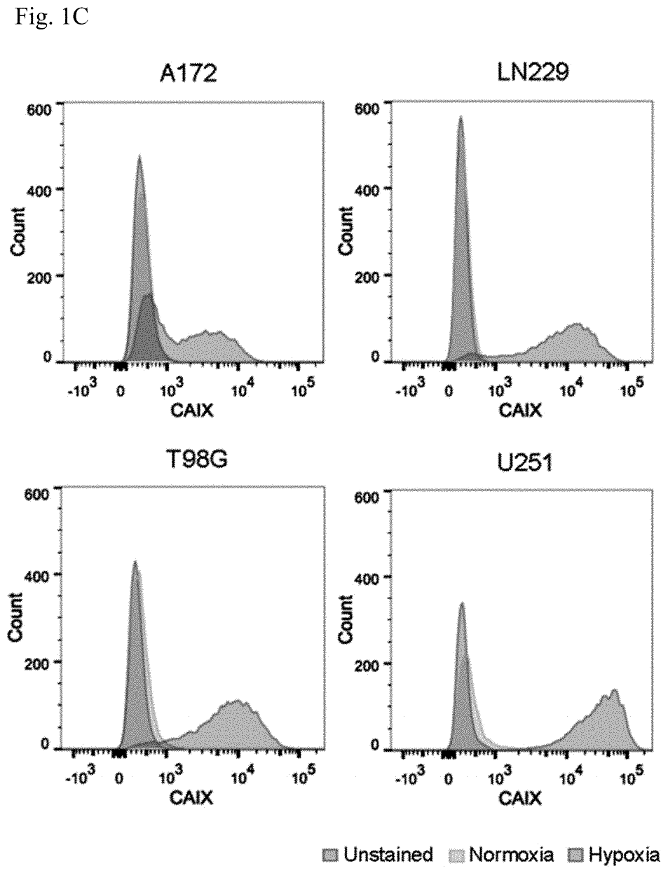

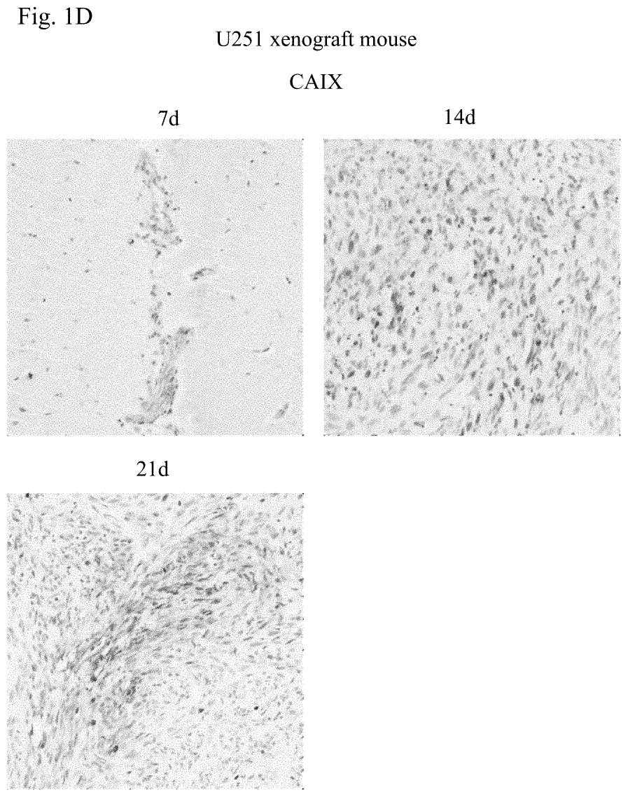

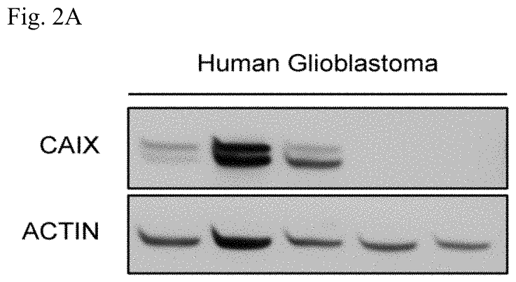

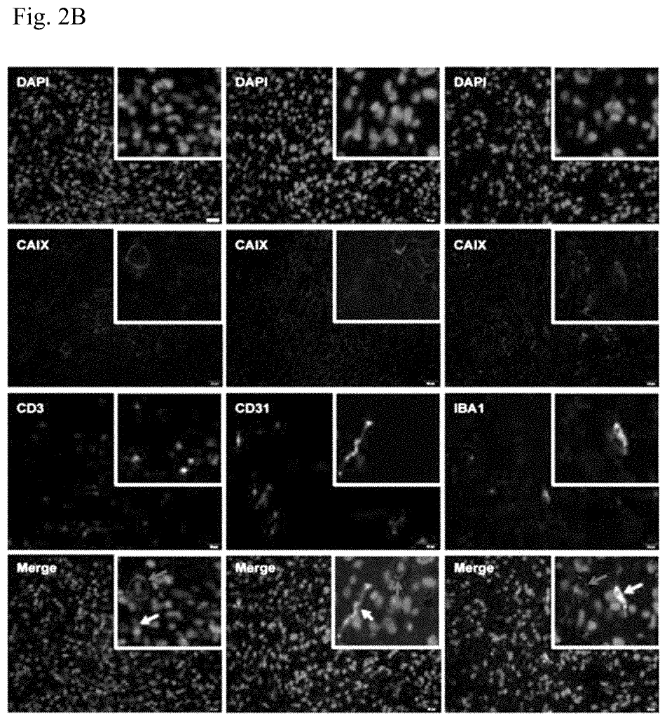

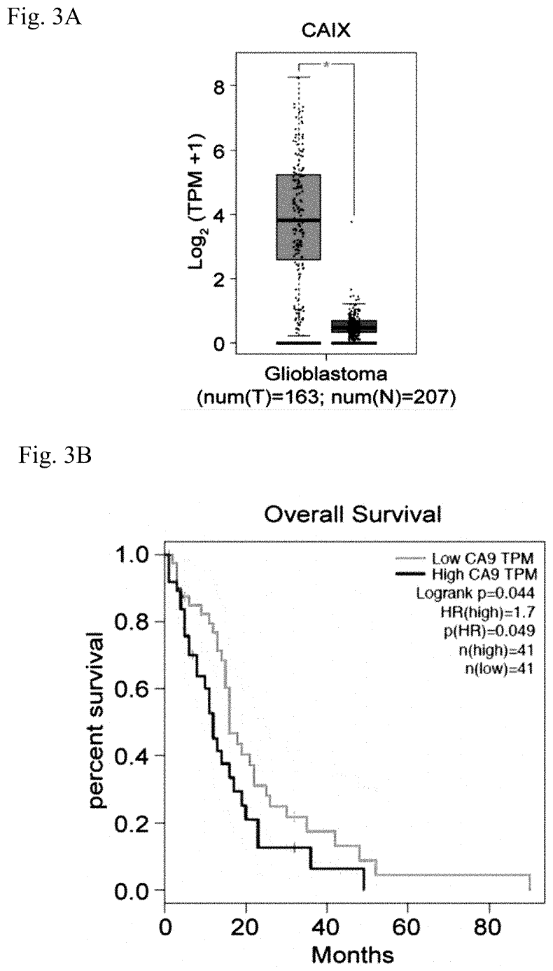

In order to prove that CAIX is a potential target for GBM, a study was conducted to test the expression of CAIX in three grade III and five grade IV glioma samples. Three out of five GBM (grade IV glioma) samples were detected with overexpression of CAIX, while no CAIX was detected in grade III gliomas (FIG. 1A). A further study was tested to confirm the results with another 27 resected GBM samples and 18 of them were positive for CAIX staining (FIG. 2A). Consistently, a large cohort of samples from the TCGA database demonstrated a dramatic up-regulation of CAIX transcription in GBM tissues compared to that in the relatively normal tissues (FIGS. 3A and 3B). The high frequency of CAIX overexpression and the significantly poor prognosis of patients with high CAIX transcription suggested that CAIX might be a promising target for GBM treatment.

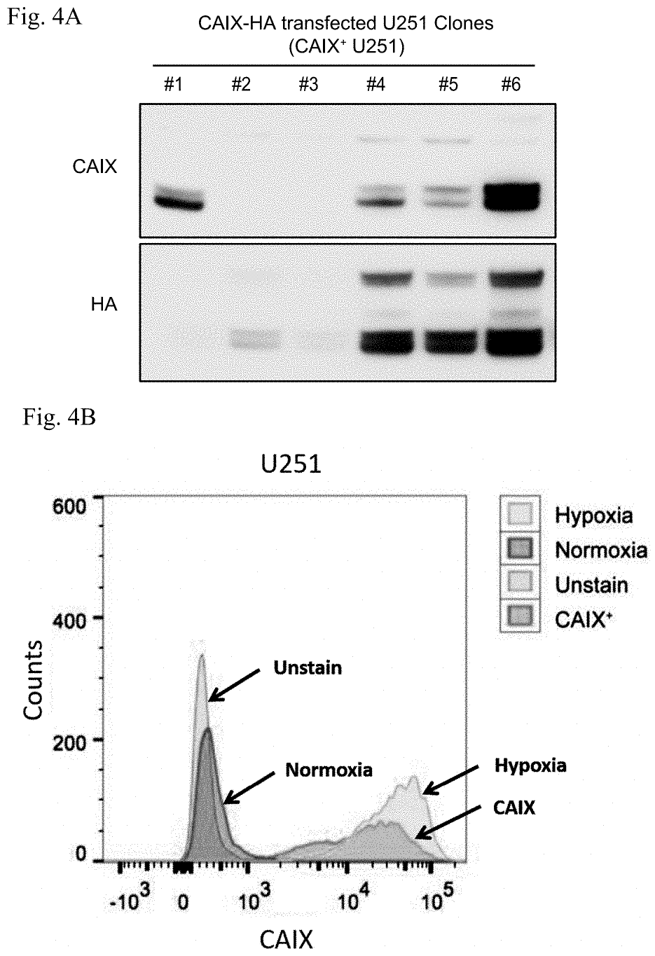

However, GBM cell lines cultured in vitro normally express low levels of CAIX, but can be significantly induced by hypoxia (FIGS. 1B and 1C). In a study to test the efficacy of CAIX CAR-T cells in vitro, naïve and endogenous CAIX transfected GBM cell lines (FIGS. 4A and 4B were used, which were confirmed with high expression of CAIX on cell membrane (FIGS. 4A and 4B).

### Generation of CAIX CAR-T

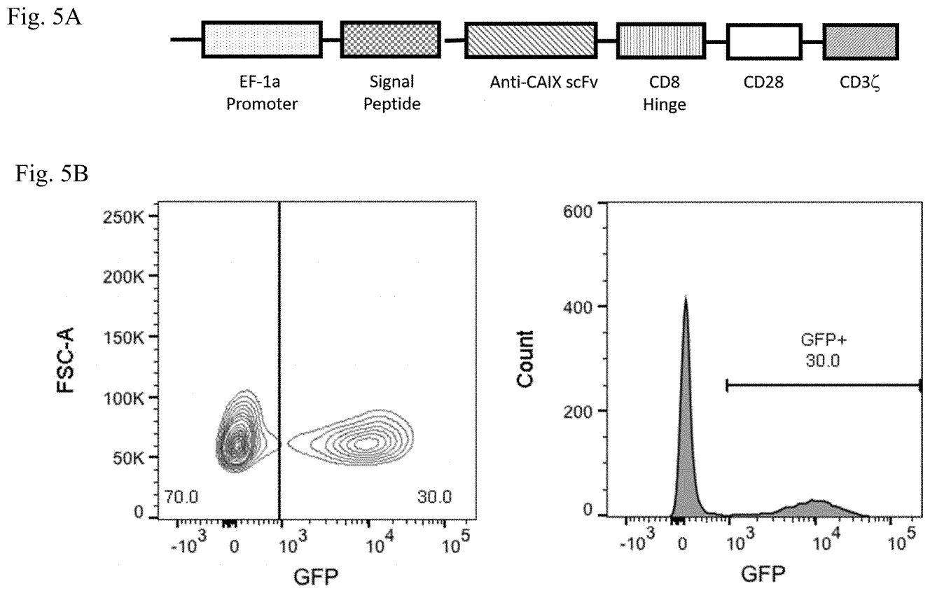

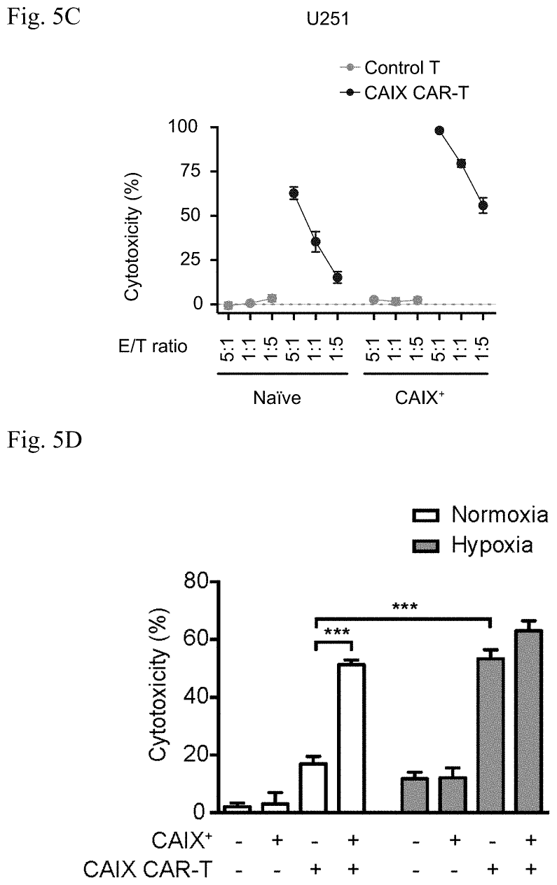

A 3^{rd} generation CAR-T vector (FIG. 5A) was transduced into donor-derived T cells. Flow cytometry showed that about 30% T cells expressed CAR (FIG. 5B).

### CAIX CAR-T Shows Specific Cytotoxicity In Vitro

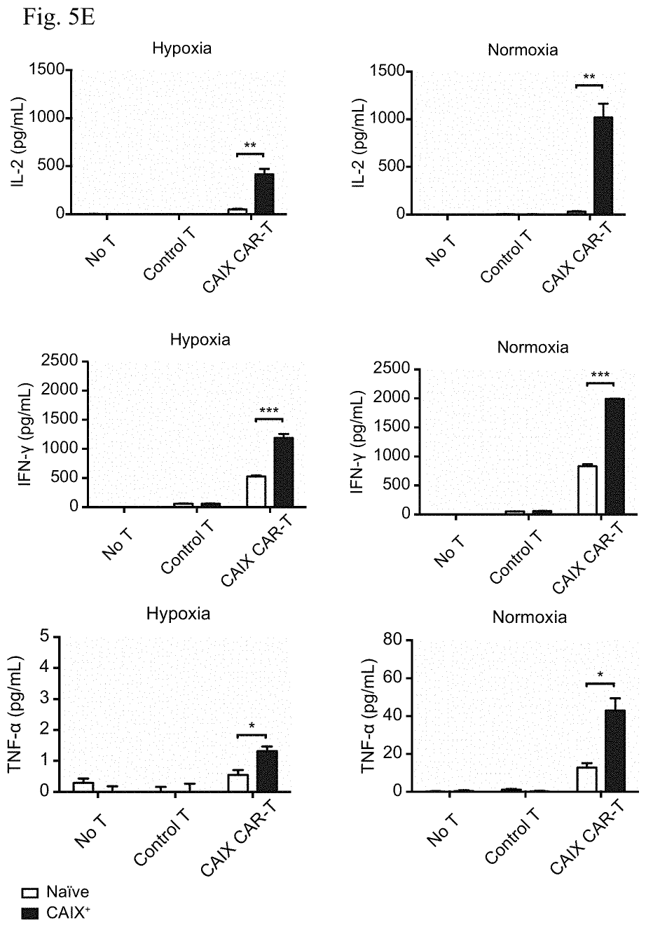

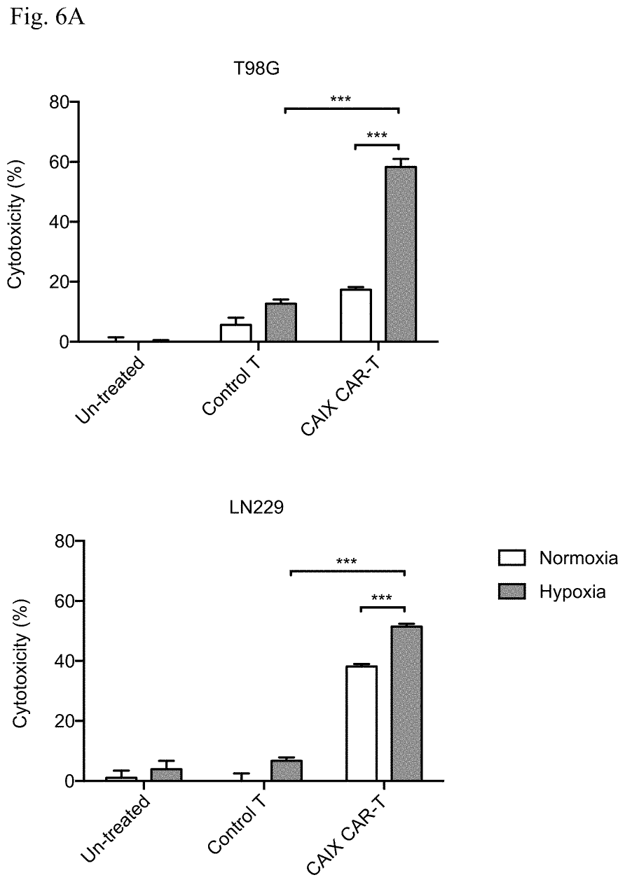

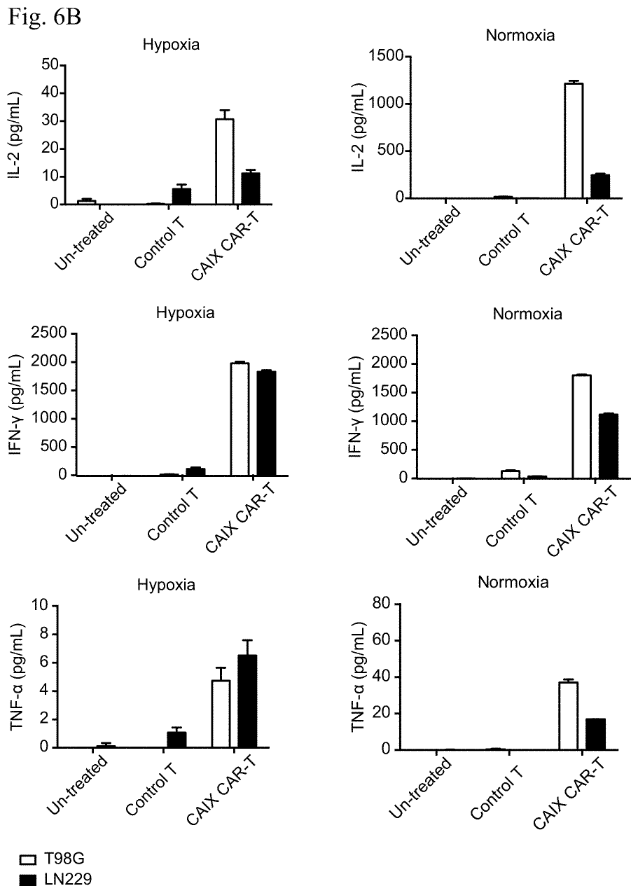

First, the specific cytotoxicity of the CAIX CAR-T cells was tested using naïve and CAIX-transfected U251 cells, and noticed that CAR-T cells manifested an impressive cytotoxicity on CAIX cells but had merely a little effect on naïve U251 cells (FIGS. 5C and 5D). Then, different numbers of CAIX CAR-T cells and non-transfected control T cells were used to co-culture with CAIX-transfected U251 cells at a constant effector/tumor (E/T) ratio of 4. A high number of cells showed much more significant cytotoxicity of CAR-T cells (FIGS. 5C and 5D), suggesting an important role of antigen density in the efficacy of CAIX CAR-T. Additionally, a hypoxia-induced model was used, which is more physiological to mimic the in vivo induction of CAIX expression, to test the efficacy of CAIX CAR-T cells. A significantly enhanced cytotoxicity of CAR-T cells on naïve U251 cells exposed to hypoxia (FIG. 5D) was observed. Similar results were observed in LN229 and T98G cells (FIGS. 6A and 6B).

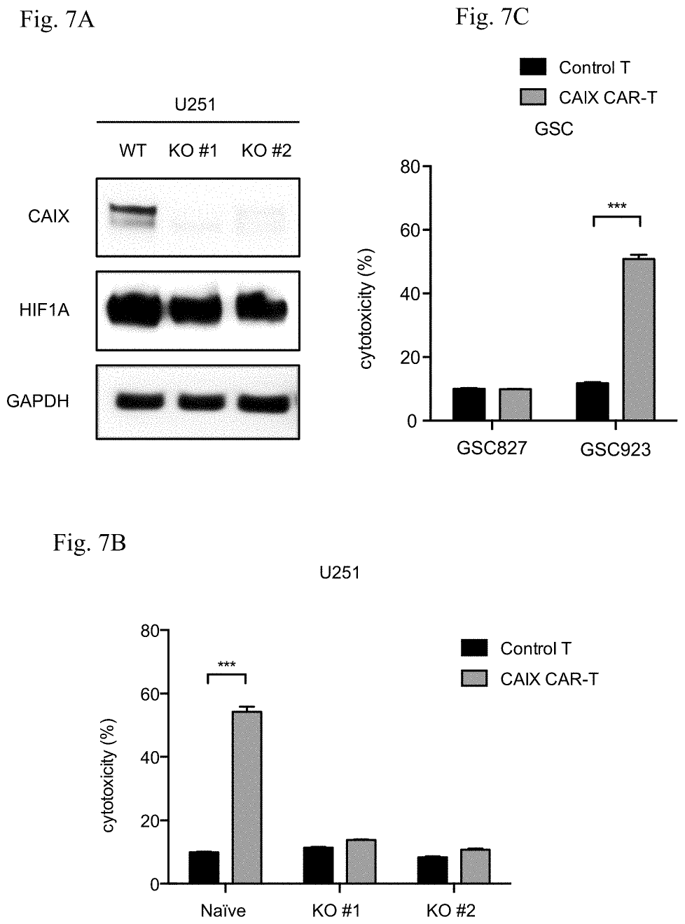

To verify these effects were CAIX mediated, we knocked out the CAIX gene in U251 cells, in which hypoxia was unable to induced CAIX expression (FIGS. 7A-7C). As expected, anti-CAIX CAR-T cells failed to kill the CAIX deficient cells even when they were pre-exposed to hypoxia (FIGS. 7A-7C). Consistently, GSC827 which was insensitive to CAIX expression induction showed little response to anti-CAIX CAR-T cells, while GSC923 which overexpressed CAIX in hypoxia demonstrated good response to our CAR-T therapy (FIGS. 7A-7C). Together, these results indicated that functional activation of CAR-T cells was associated with their cytotoxicity in the presence of CAIX antigen.

Consistent with its cytotoxicity of CAR-T cells, an increase levels of IFN-γ, TNF-α, and IL-2 were observed in the presence of CAIX CAR-T cells but not control T cells (no vector transfected) or mock T cells (backbone vector transfected) (FIG. 5E). Furthermore, the secretion of these cytokines was significantly up-regulated in CAIX-transfected (FIG. 5E) or hypoxia-exposed GBM cells (FIG. 6B). These results indicated that functional activation of CAR-T cells was associated with their cytotoxicity in the presence of CAIX antigen.

### CAIX CAR-T Demonstrates Anti-Tumoral Effects In Vivo

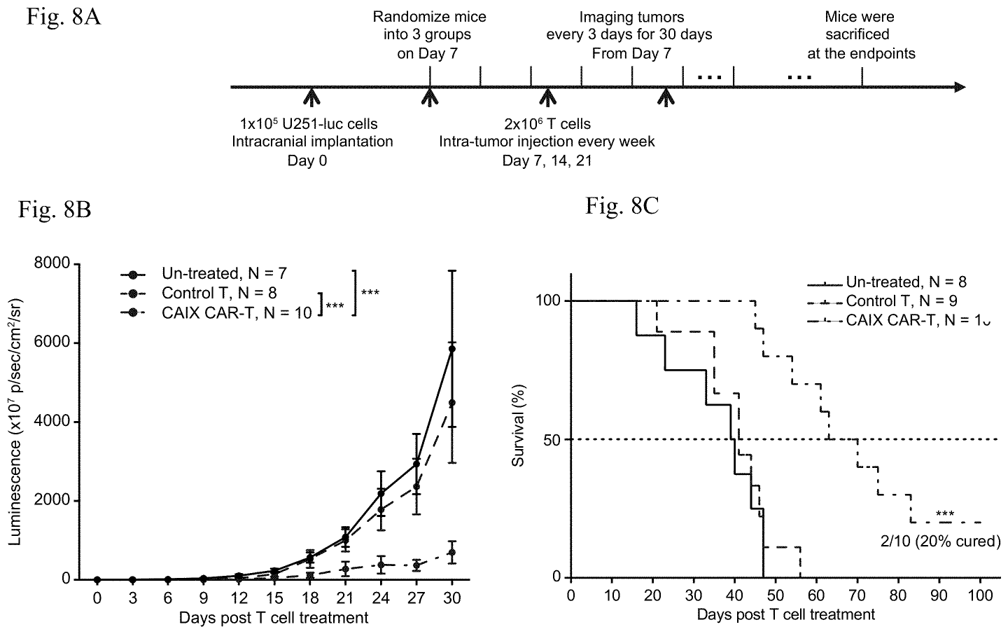

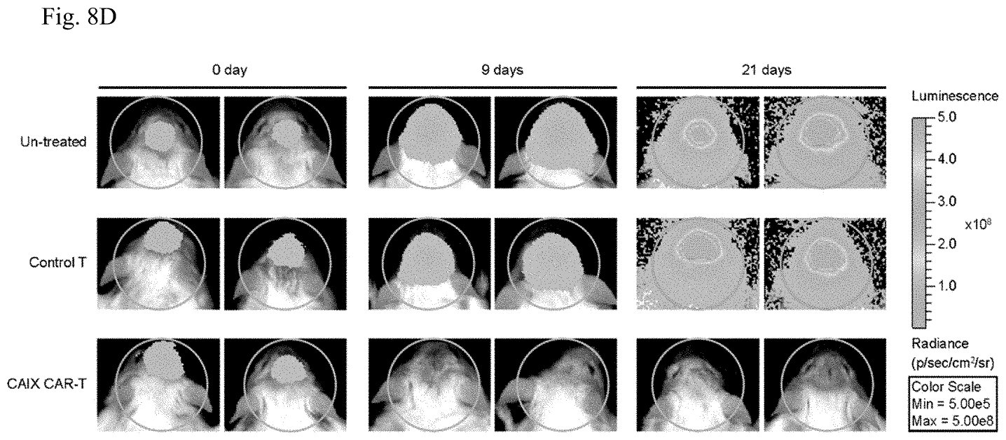

To further validate the efficacy of the CAIX CAR-T in vivo, an intracranial mouse model was generated (FIG. 8A). After around one week of inoculation of U251-luc cells, CAR-T was injected in situ. Compared to those with vehicle injection, CAIX CAR-T cells significantly limited the growth of tumor and prolonged the survival of these mice (FIGS. 8B and 8C). To enhance the efficacy, a multi-injection strategy was tried in the mouse model and the CAR-T group demonstrated a significant delay in tumor growth (FIGS. 8B and 8C). Strikingly, CAIX CAR-T cells cured two out of ten initially treated mice, while control T cells did not show such an efficacy (FIG. 8D).

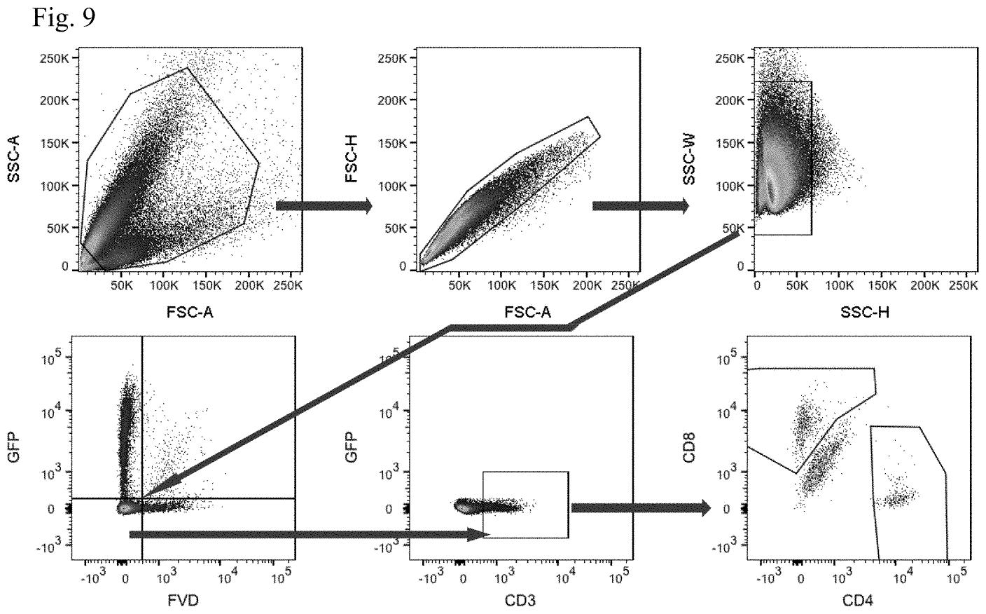

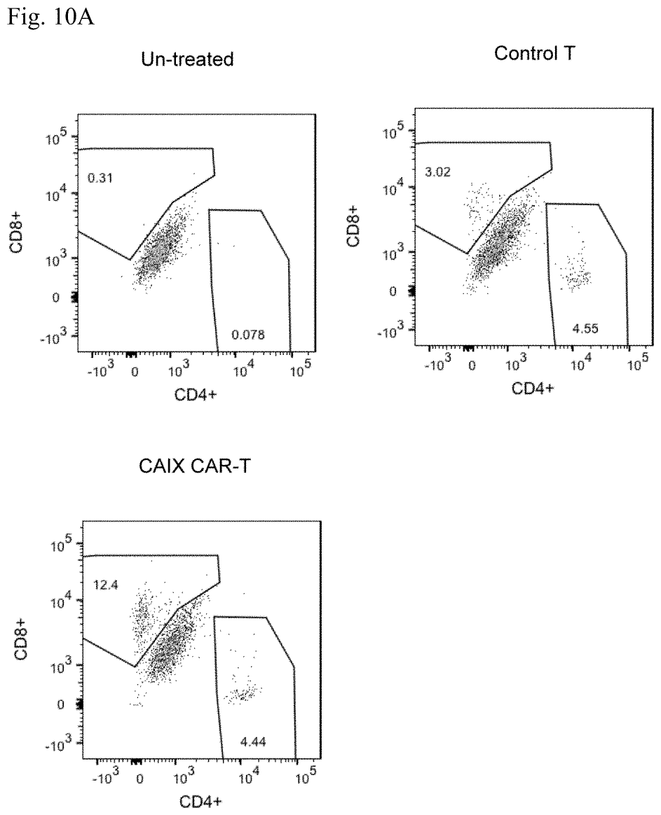

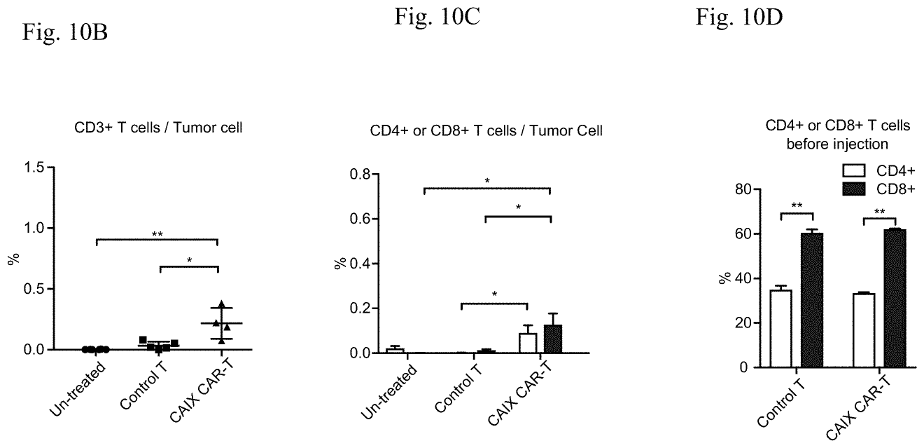

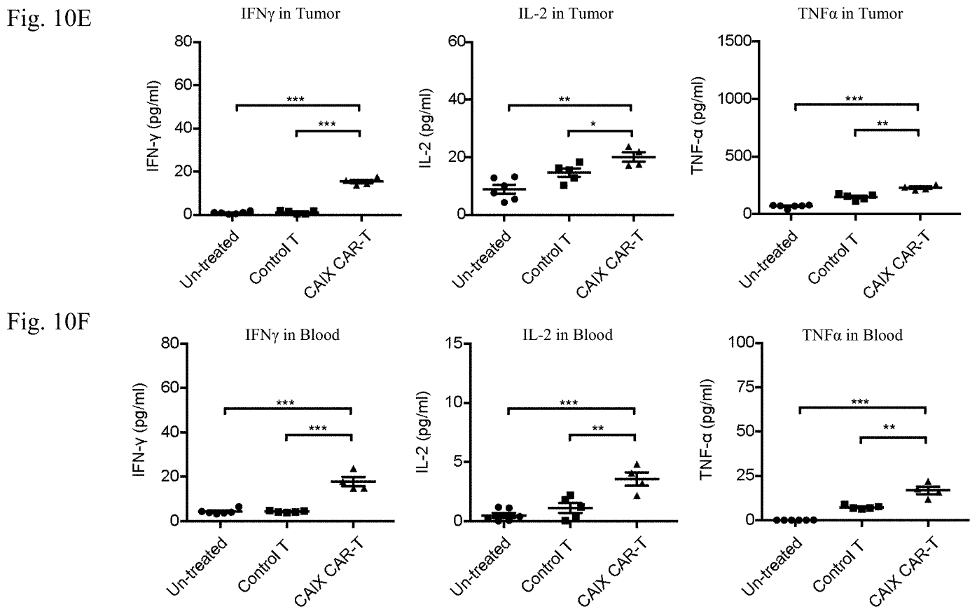

Pathological analysis of the tumor cells revealed significant infiltration of immune cells and release of cytokines in tumors with CAR-T injection. Two-weeks after treatment, the brain tumors were harvested and analyzed by flow cytometry with human T cell markers (CD3, CD4, and CD8) following the gating strategy showed in FIG. 9. CAR-T cell response to CAIX antigen was evaluated by tumor infiltrating lymphocytes (TILs) analysis. In brain tumors, we observed an increase in CD3+ T cells in mice treated with anti-CAIX CAR-T cells (FIG. 10A). In particular, treatment with anti-CAIX CAR-T cells resulted in a significant increase in the abundance of both CD8+ T cells and CD4+ T cells (FIGS. 10B-10D). This indicated that CAR-T cells gained stronger survival and/or proliferation abilities upon the stimulation of CAIX antigen. Notably, cytotoxic CD8+ T cells had a better advantage in survival and/or proliferation compared CD4+ T cells before injection (FIG. 10D). In addition, we also observed a similar trend in the secretion of IFN-γ, TNF-α, and IL-2 within tumor supernatant and blood where treatment with anti-CAIX CAR-T cells showed an increase in cytokine secretion (FIGS. 10E and 10F).

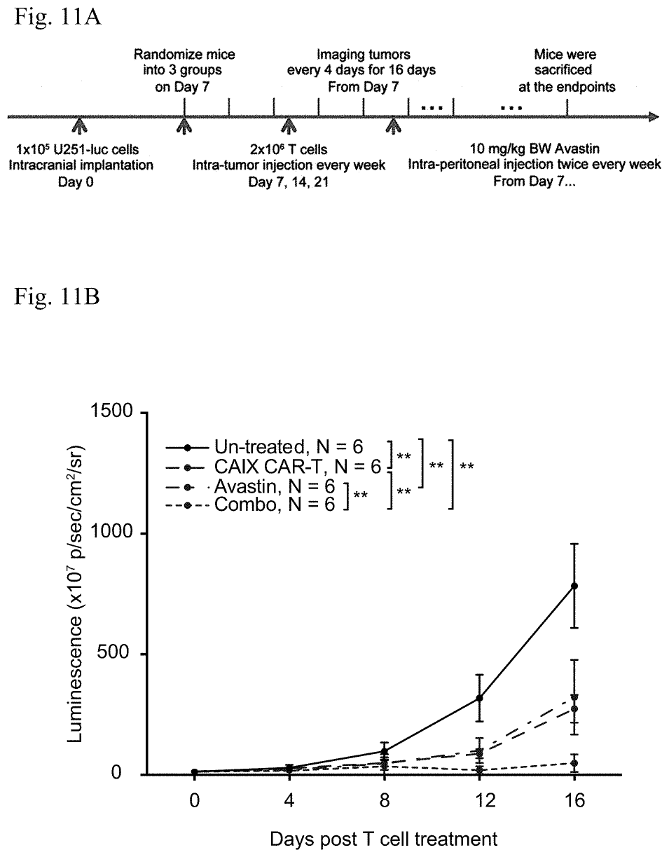

In addition, to take advantage of the natural hypoxia caused by glioblastoma progression, the efficacy of anti-CAIX CAR-T cells may be further increased by pharmacologic induction of hypoxia in tumor microenvironment using anti-angiogenic agents such as Avastin and sorafenib. We found that the combination of Avastin and anti-CAIX CAR-T had a syngeneic anti-tumor effect superior to either therapy alone (FIGS. 11A and 11B).

The use of the word “a” or “an” when used in conjunction with the term “comprising” in the claims and/or the specification may mean “one,” but it is also consistent with the meaning of “one or more,” “at least one,” and “one or more than one.”

Throughout this application, the term “about” is used to indicate that a value includes the standard deviation of error for the device or method being employed to determine the value.

The use of the term “or” in the claims is used to mean “and/or” unless explicitly indicated to refer to alternatives only or the alternatives are mutually exclusive, although the disclosure supports a definition that refers to only alternatives and “and/or.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed. To the extent a definition of a term set out in a document incorporated herein by reference conflicts with the definition of a term explicitly defined herein, the definition set out herein controls.

What is claimed is:

1. A method for treating glioblastoma or other brain tumors, the method comprising:
preparing cells comprising a chimeric antigen receptor (CAR) molecule, the CAR molecule having an antigen binding domain that binds to the tumor antigen associated with the glioblastoma or other brain tumors, and administering to a mammal in need thereof an effective amount of the prepared cells,
wherein:
the tumor antigen comprises carbonic anhydrase IX;
the administering of the prepared cells is through intracranial injection; and
the intracranial injection is directly targeting within a boundary of the glioblastoma or the other brain tumors.

2. The method of claim 1, wherein the intracranial injection is conducted stereotactically.

3. The method of claim 1, wherein the prepared cells further comprise an agent for use in combination with the CAR molecule to increase the efficacy of the treatment.

4. The method of claim 3, wherein the agent comprises a molecule stimulating lymphocyte proliferation.

5. The method of claim 4, wherein the molecule stimulating lymphocyte proliferation comprises interleukin 7.

6. The method of claim 3, wherein the agent comprises a molecule recruiting through chemotaxis endogenous immune cells to eliminate glioblastoma or other brain tumors.

7. The method of claim 6, wherein the molecule recruiting through chemotaxis endogenous immune cells comprises chemokine (C-C motif) ligand 19.

8. The method of claim 1, further comprising, before the administering of the prepared cells, treating the glioblastoma or the other brain tumors with a therapy that inhibits vascular endothelial growth factor.

9. The method of claim 8, wherein the therapy that inhibits vascular endothelial growth factor comprises administering an effective amount of Avastin.

10. The method of claim 9, wherein the mammal is a human.
